(19) United States
(12) Patent Application Publication
(10) Pub. No.: US 2021/0158915 A1
(43) Pub. Date: May 27, 2021

(54) REGENERATIVE MEDICINE SUPPORT SYSTEM, REGENERATIVE MEDICINE SUPPORT METHOD, AND REGENERATIVE MEDICINE SUPPORT PROGRAM

(71) Applicant: FUJIFILM CORPORATION, Tokyo (JP)

(72) Inventors: Takeshi FUKUDA, Kanagawa (JP); Haruyasu NAKATSUGAWA, Kanagawa (JP)

(21) Appl. No.: 16/950,925

(22) Filed: Nov. 18, 2020

(30) Foreign Application Priority Data
Nov. 21, 2019 (JP) 2019-210785

(51) Int. Cl. G16H 10/60; G06N 20/00

(52) U.S. Cl. CPC G16H 10/60; G06N 20/00

(57) ABSTRACT

Provided are a regenerative medicine support system, a regenerative medicine support method, and a regenerative medicine support program capable of obtaining an appropriate treatment policy according to a target animal that is a target of regenerative medicine. The regenerative medicine support system includes a CPU and a storage unit that stores a command executable by the CPU in an information processing apparatus. The CPU collects a plurality of pieces of treatment record information including content of treatment based on a regenerative medicine and a treatment result by the treatment for each breed of an animal that is a target of the treatment, acquires breed information indicating the breed of the target animal that is the target of the treatment, and provides a treatment policy of the target animal, which is derived on the basis of the treatment record information in which the breed represented by the breed information is the treatment target among the plurality of pieces of collected treatment record information.

### CROSS-REFERENCE TO RELATED APPLICATIONS

The present application claims priority under 35 U.S.C. § 119 to Japanese Patent Application No. 2019-210785, filed on Nov. 21, 2019. The above application is hereby expressly incorporated by reference, in its entirety, into the present application.

### BACKGROUND

### 1. Technical Field

The present disclosure relates to a regenerative medicine support system, a regenerative medicine support method, and a regenerative medicine support program.

### 2. Description of the Related Art

A technique for supporting medical care of a patient is generally disclosed. For example, JP2016-162131A discloses a technique for presenting a recommended drug or searching for similar cases on the basis of patient medical care data and the like.

### SUMMARY

By the way, in recent years, treatment based on regenerative medicine has increasingly performed, and regenerative medicine that targets animals has also been performed. However, a technology for supporting regenerative medicine has not been sufficiently developed. In particular, in a case where a treatment target is an animal, an appropriate regenerative medicine tends to differ depending on a breed of the target animal, and thus, it may be difficult to obtain an appropriate treatment policy in the regenerative medicine.

The present disclosure has been made in view of the above circumstances, and provides a regenerative medicine support system, a regenerative medicine support method, and a regenerative medicine support program capable of obtaining an appropriate treatment policy according to a target animal that is a target of regenerative medicine.

In order to achieve the above object, according to a first aspect of the present disclosure, there is provided a regenerative medicine support system comprising: at least one processor; and a memory that stores a command executable by the processor, in which the processor collects a plurality of pieces of treatment record information including content of treatment based on a regenerative medicine and a treatment result by the treatment for each breed of an animal that is a target of the treatment, obtains breed information indicating the breed of the target animal that is the target of the treatment, and provides a treatment policy of the target animal, which is derived on the basis of the treatment record information in which the breed represented by the breed information is the treatment target among the plurality of pieces of collected treatment record information.

According to a second aspect of the present disclosure, in the regenerative medicine support system according to the first aspect of the present disclosure, the processor provides, in a case where the treatment record information in which the breed represented by the breed information is the treatment target is not included in the plurality of pieces of collected treatment record information, the treatment policy of the target animal, which is derived on the basis of the treatment record information in which a closely related breed of the breed represented by the breed information, instead of the breed represented by the breed information, is set as the treatment target.

According to a third aspect of the present disclosure, in the regenerative medicine support system according to the second aspect of the present disclosure, the closely related breed is a breed of at least one of parents of the target animal.

According to a fourth aspect of the present disclosure, in the regenerative medicine support system according to the second aspect of the present disclosure, the closely related breed is a breed that is determined to be closely related to the breed of the target animal on the basis of genes.

According to a fifth aspect of the present disclosure, in the regenerative medicine support system according to the first aspect of the present disclosure, the content of the treatment and the treatment policy include at least one of information relating to the type of cells used for the treatment, information relating to administration of the cells, or information relating to surgery.

According to a sixth aspect of the present disclosure, in the regenerative medicine support system according to the first aspect of the present disclosure, the processor performs the derivation of the treatment policy on the basis of a learned model learned in advance using the plurality of pieces of treatment record information and the breed represented by the breed information.

Further, in order to achieve the above object, according to a seventh aspect of the present disclosure, there is provided a regenerative medicine support system comprising: an information processing apparatus including at least one processor and a memory that stores a command executable by the processor; and a terminal device communicably connected to the information processing apparatus, wherein the processor collects a plurality of pieces of treatment record information including content of treatment based on a regenerative medicine and a treatment result by the treatment for each breed of an animal that is a target of the treatment, acquires breed information indicating the breed of the target animal that is the target of the treatment, and provides a treatment policy of the target animal, which is derived on the basis of the treatment record information in which the breed represented by the breed information is the treatment target among the plurality of pieces of collected treatment record information, and wherein the terminal device performs display based on the treatment policy provided from the information processing device.

In addition, in order to achieve the above object, according to an eighth aspect of the present disclosure, there is provided a regenerative medicine support method executed by a computer, the method comprising: collecting a plurality of pieces of treatment record information including content of treatment based on a regenerative medicine and a treatment result by the treatment for each breed of an animal that is a target of the treatment; acquiring breed information indicating the breed of the target animal that is the target of the treatment; providing a treatment policy of the target animal, which is derived on the basis of the treatment record information in which the breed represented by the breed information is the treatment target among the plurality of pieces of collected treatment record information; and performing display based on the provided treatment policy.

Furthermore, in order to achieve the above object, a non-transitory computer-readable storage medium storing according to a ninth aspect of the present disclosure, there is provided a regenerative medicine support program for collecting a plurality of pieces of treatment record information including content of treatment based on a regenerative medicine and a treatment result by the treatment for each breed of an animal that is a target of the treatment; acquiring breed information indicating the breed of the target animal that is the target of the treatment; and providing a treatment policy of the target animal, which is derived on the basis of the treatment record information in which the breed represented by the breed information is the treatment target among the plurality of pieces of collected treatment record information.

According to the present disclosure, it is possible to obtain an appropriate treatment policy according to a target animal that is a target of regenerative medicine.

### BRIEF DESCRIPTION OF THE DRAWINGS

Exemplary embodiments according to the technique of the present disclosure will be described in detail based on the following figures, wherein: FIG. 1 is a block diagram showing an example of a configuration of a regenerative medicine support system.

FIG. 2 is a block diagram illustrating an example of a hardware configuration of an information processing apparatus.

FIG. 3 is a diagram for explaining an example of learning information, breed information, and treatment record information.

FIG. 4 is a diagram for explaining an example of a learned model.

FIG. 5 is a block diagram showing an example of a functional configuration in a treatment record information collecting phase of the information processing apparatus.

FIG. 6 is a flowchart showing an example of a treatment record information collecting process executed by the information processing apparatus.

FIG. 7 is a block diagram showing an example of a functional configuration in a learning phase of the information processing apparatus.

FIG. 8 is a diagram for explaining an example of input/output of a learned model.

FIG. 9 is a flowchart showing an example of a learning process executed by the information processing apparatus.

FIG. 10 is a block diagram showing an example of a functional configuration in an operation phase of the information processing apparatus.

FIG. 11 is a flowchart showing an example of a treatment policy providing process executed by the information processing apparatus.

FIG. 12 is a diagram for explaining derivation of a treatment policy using a learned model according to a disease or an injury in the information processing apparatus.

FIG. 13 is a block diagram showing an example of a hardware configuration of a terminal device.

FIG. 14 is a flowchart showing an example of a treatment policy display process executed by the terminal device.

FIG. 15 is a diagram showing an example of display of a treatment policy executed by the terminal device.

FIG. 16 is a diagram showing an example of learning information stored in a storage unit of an information processing apparatus according to a modified example.

FIG. 17 is a diagram for explaining an example of a learned model according to a modified example.

FIG. 18 is a diagram for explaining an example of a learned model according to a modified example.

FIG. 19 is a diagram for explaining an example of input/output of a learned model according to a modified example.

FIG. 20 is a diagram for explaining derivation of a treatment policy using a learned model according to a disease or an injury in the information processing apparatus according to a modified example.

### DETAILED DESCRIPTION

Hereinafter, an exemplary embodiment for carrying out the technique of the present disclosure will be described in detail with reference to the accompanying drawings. In addition, in the following embodiments, a case where a “dog” is applied as a specific example of an animal that is a treatment target and a target animal that is a regenerative medicine target will be described.

[Regenerative Medicine Support System]

A regenerative medicine support system 1 of the present embodiment will be described with reference to FIG. 1. FIG. 1 is a block diagram showing an example of a configuration of the regenerative medicine support system 1 of the present embodiment. As shown in FIG. 1, the regenerative medicine support system 1 of the present embodiment includes an information processing apparatus 10 and a plurality of (in FIG. 1, three as an example) terminal devices 12. The information processing apparatus 10 and the plurality of terminal devices 12 are respectively connected to a network N, and are able to communicate with each other through the network N.

The information processing apparatus 10 is, for example, a cloud server constructed on a cloud. The information processing apparatus 10 may be a server computer installed in a veterinary hospital or the like. The terminal device 12 is installed in, for example, a veterinary hospital, and is used by a user such as a veterinarian. Examples of the terminal device 12 include a personal computer, a tablet computer, or the like.

[Information Processing Apparatus]

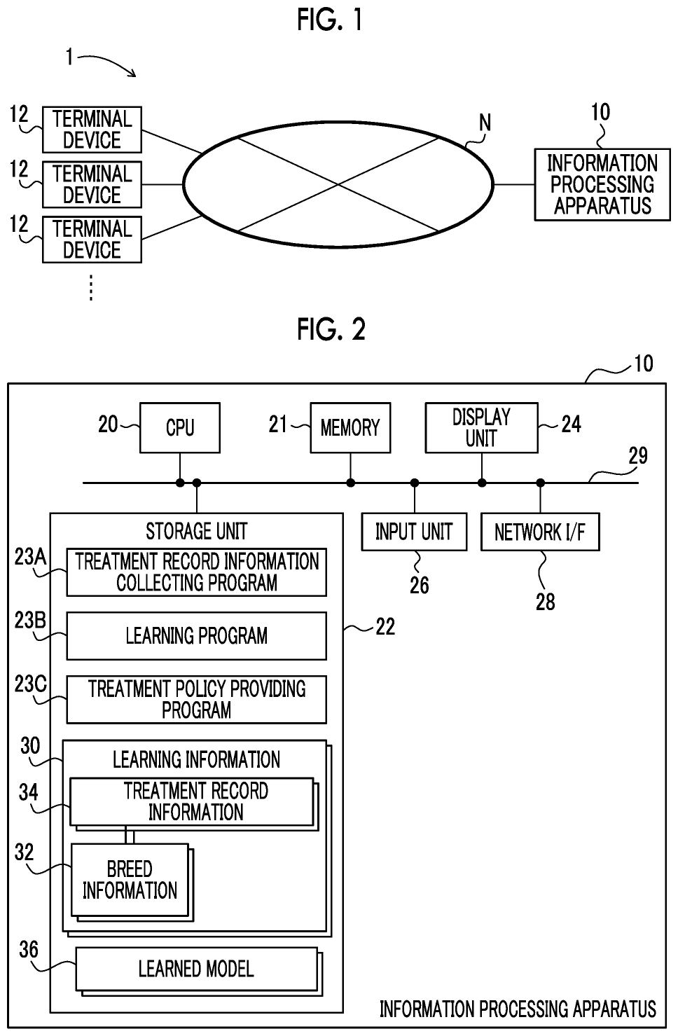

An example of a hardware configuration of the information processing apparatus 10 according to the present embodiment will be described with reference to FIG. 2. As shown in FIG. 2, the information processing apparatus 10 includes a central processing unit (CPU) 20, a memory 21 as a temporary storage area, and a non-volatile storage unit 22. The storage unit 22 is an example of a memory according to the present disclosure.

Further, the information processing apparatus 10 includes a display unit 24 such as a liquid crystal display, an input unit 26 such as a keyboard and a mouse, and a network interface (I/F) 28 connected to the network N. The display unit 24 and the input unit 26 may be integrated as a touch panel display. The CPU 20, the memory 21, the storage unit 22, the display unit 24, the input unit 26, and the network I/F 28 are connected to a bus 29 to communicate with each other.

The storage unit 22 is realized by a hard disk drive (HDD), a solid state drive (SSD), a flash memory, or the like. The storage unit 22 as a storage medium stores a treatment record information collecting program 23A. The CPU 20 reads out the treatment record information collecting program 23A from the storage unit 22, expands the treatment record information collecting program 23A in the memory 21, and executes the expanded treatment record information collecting program 23A. Further, the storage unit 22 stores a learning program 23B. The CPU 20 reads out the learning program 23B from the storage unit 22, expands the learning program 23B in the memory 21, and executes the expanded learning program 23B. Further, the storage unit 22 stores a treatment policy providing program 23C. The CPU 20 reads out the treatment policy providing program 23C from the storage unit 22, expands the treatment policy providing program 23C in the memory 21, and executes the expanded the treatment policy providing program 23C.

Further, the storage unit 22 of the present embodiment stores learning information 30 and a learned model 36 learned using the learning information 30. In the present embodiment, the learned model 36 and the learning information 30 are stored for each disease or injury.

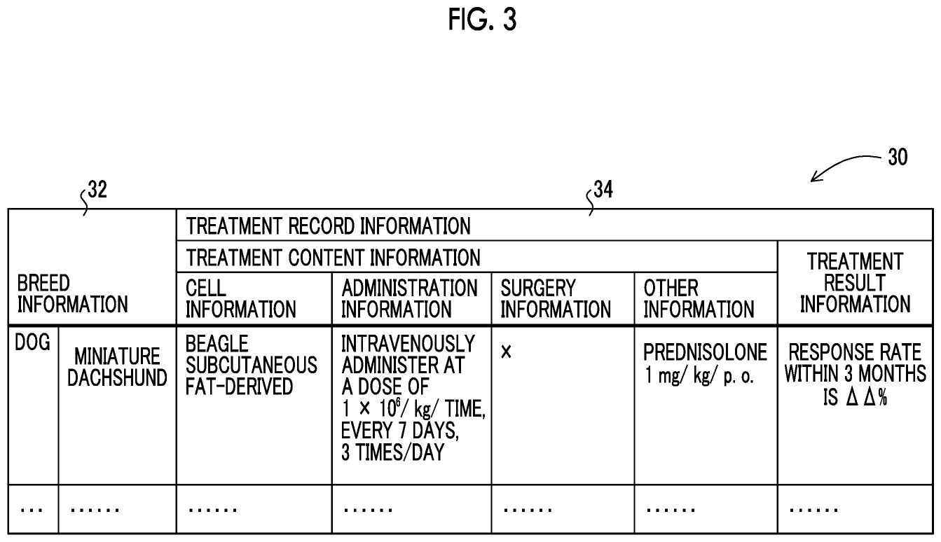

As shown in FIGS. 2 and 3, as an example, the learning information 30 according to the present embodiment includes breed information 32 and treatment record information 34.

The breed information 32 is breed information indicating a breed of a dog that is a target of treatment based on regenerative medicine. Specifically, since the animal is a dog, the breed information 32 is information indicating a dog breed. In the present embodiment, the term “breed” includes not only a breed such as a “dog breed” but also a concept of a race such as a “dog” and a “cat”.

As shown in FIG. 3, the treatment record information 34 includes treatment content information and treatment result information. The treatment content information is information indicating content of treatment based on regenerative medicine, in other words, information indicating content of treatment performed for a dog that is a target of treatment. As an example, the treatment content information of this embodiment includes cell information, administration information, surgery information, and other information. The cell information is information indicating the type of cells used for treatment, and in the present embodiment, is information indicating the type of used cells and a derived site thereof. The administration information is information relating to administration of cells, and in the present embodiment, is information indicating the dose of administration, the number of administrations, an administration period, and an administration route of used cells. The surgery information is information relating to surgery, and in this embodiment, is information on a surgery performed in combination with regenerative medicine. Further, the other information is information relating to treatment other than the above-mentioned information, and in the present embodiment, is information on cotreatment performed in combination with regenerative medicine, which is information on cotreatment other than surgery.

On the other hand, the treatment result information is information indicating a treatment result according to treatment content represented by the treatment content information, and in this embodiment, a response rate is applied as the treatment result information. In addition, in a case where an effect of treatment is not obtained, or in a case where it can be considered that the effect is not obtained, information indicating “ineffective” is recorded as the treatment result information.

For example, in the example shown in FIG. 3, an example of the learning information 30 for “chronic enteropathy” is shown. The example shown in FIG. 3 shows a treatment in which cells of “subcutaneous fat-derived” of “Beagle” are administered to “Miniature Dachshund” at a dose of “10⁶ cells/kg per body weight (1×10⁶/kg)”, “every 7 days”, “3 times a day (3 times/day)” intravenously and “prednisolone” is “orally administered at a dose of 1 mg/kg per body weight (1 mg/kg p. o.)” is performed. In addition, the treatment result indicates that “a response rate within 3 months was 44%”.

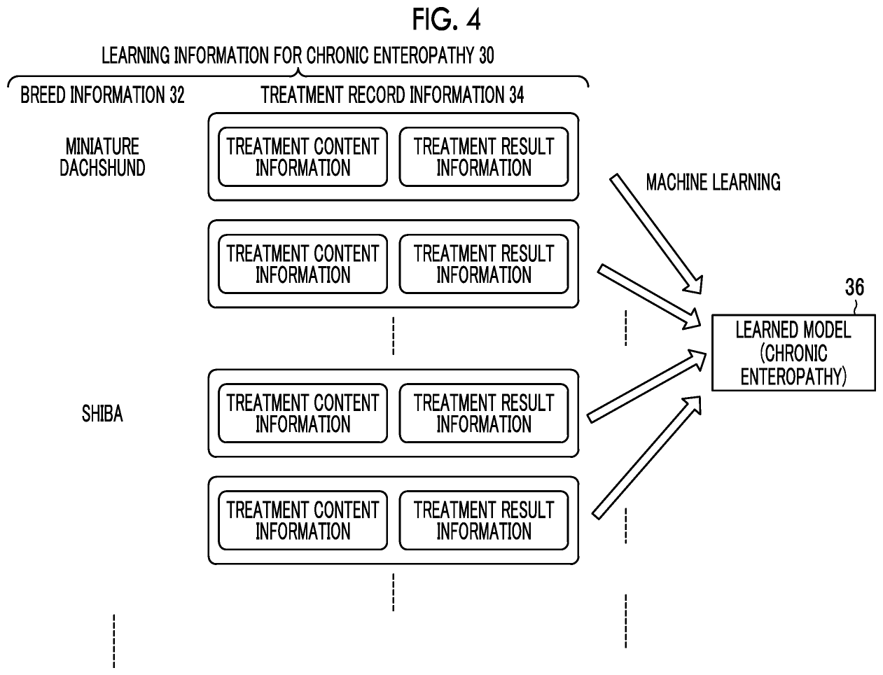

The learned model 36 is a model learned using the learning information 30. In the present embodiment, as an example, as shown in FIG. 4, the learned model 36 is generated by machine learning using the learning information 30. For example, as shown in FIG. 4, the learned model 36 for chronic enteropathy is generated from the learning information 30 for the chronic enteropathy including the treatment record information 34 that the dog breed represented by the breed information 32 is “Miniature Dachshund”, the treatment record information 34 that the dog breed represented by the breed information 32 is “Shiba”, and the like. An example of the learned model 36 includes a neural network model.

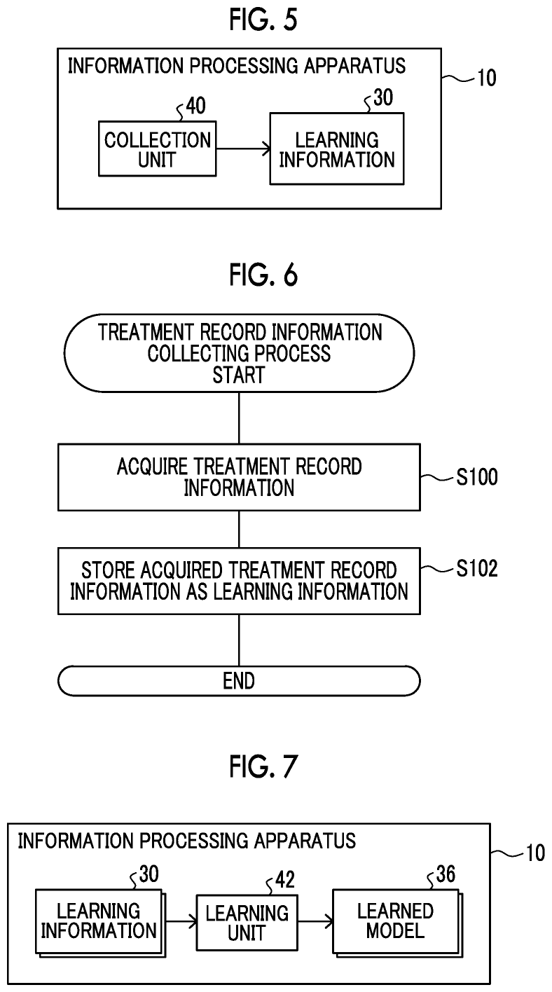

Next, a functional configuration of the information processing apparatus 10 of the present embodiment in a treatment record information collecting phase will be described with reference to FIG. 5. As shown in FIG. 5, the information processing apparatus 10 includes a collection unit 40. The CPU 20 functions as the collection unit 40 by executing the treatment record information collecting program 23A.

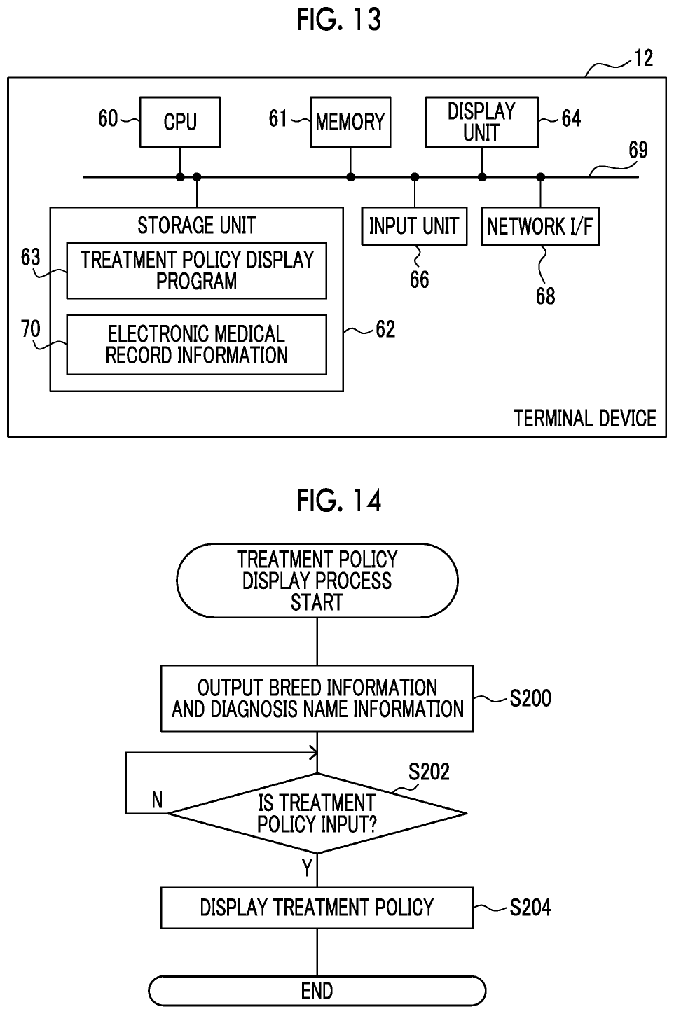

The collection unit 40 collects the treatment record information 34 for each breed. In the terminal device 12, treatment record information relating to treatment performed in a veterinary hospital in which the terminal device 12 is installed is stored as electronic medical record information (electronic medical record information 70, see FIG. 13). The collection unit 40 of the present embodiment accesses the terminal device 12 through the network N, and acquires the treatment record information relating to treatment based on regenerative medicine in association with information indicating a disease or injury and information indicating an animal breed that is a breed, from the electronic medical record information 70 stored in the terminal device 12, to collect the treatment record information 34.

Next, an operation of the information processing apparatus 10 of the present embodiment in the treatment record information collecting phase will be described with reference to FIG. 6. As the CPU 20 executes the treatment record information collecting program 23A, a treatment record information collecting process shown in FIG. 6 is executed. The treatment record information collecting process shown in FIG. 6 is executed for each of the terminal devices 12 included in the regenerative medicine support system 1. The treatment record information collecting process shown in FIG. 6 is executed at regular timings such as once every 3 days. The timing at which the treatment record information collecting process illustrated in FIG. 6 is performed may be different for each terminal device 12, and for example, a closing date or a closing time of the veterinary hospital in which the terminal device 12 is installed may be set as the timing at which the treatment record information collecting process is performed.

In Step S100 of FIG. 6, the collection unit 40 acquires treatment record information from the terminal device 12 as the treatment record information 34, as described above.

In the next Step S102, the collection unit 40 stores the treatment record information 34 acquired in Step S100 in the storage unit 22 as the learning information 30. In a case where the process of Step S102 ends, the treatment record information collecting process ends.

Next, a functional configuration of the information processing apparatus 10 in a learning phase of the present embodiment will be described with reference to FIG. 7. As shown in FIG. 7, the information processing apparatus 10 includes a learning unit 42. The CPU 20 functions as the learning unit 42 by executing the learning program 23B.

The learning unit 42 generates the learned model 36 that outputs a treatment policy of a target animal on the basis of the learning information 30 by causing the learning information 30 acquired from the storage unit 22 to be learned as learning data (may also be referred to as training data). Specifically, the learning unit 42 generates plural learned models 36 according to diseases or injuries, in which breed information 50 indicating the breed of the target animal is an input and information relating to the treatment policy is an output, for each disease or injury, by machine learning.

More specifically, in a case where the breed information 50 is input, the learning unit 42 causes a model to be learned so that information indicating a treatment policy with the best treatment result (highly effective treatment) for a dog breed represented by the breed information 50 is output. In a case where there is no treatment record information 34 associated with the breed information 32 of the same breed as the dog breed represented by the inputted breed information 50 in the learning information 30 used for learning, the model is learned so that information indicating that there is no treatment policy is output. By this learning, the learned model 36 is generated for each disease or injury. In the present embodiment, the information indicating the treatment policy and the information indicating that there is no treatment policy are collectively referred to as “information related to a treatment policy”.

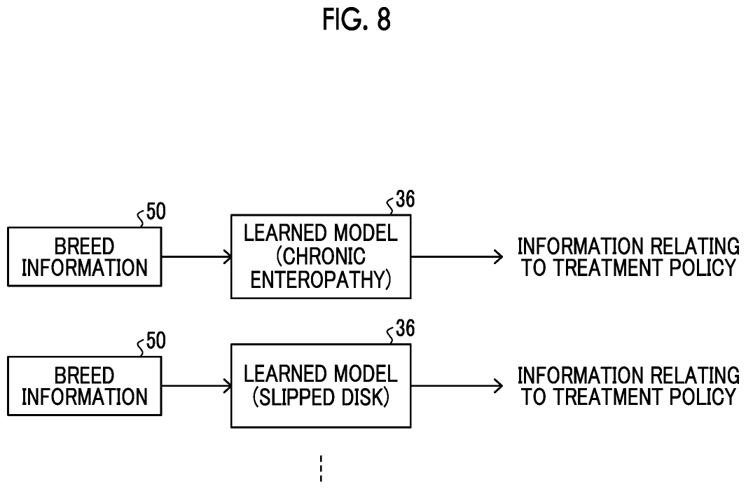

As an algorithm of the learning by the learning unit 42 described above, for example, backpropagation may be applied. By the learning by the learning unit 42 described above, as an example, as shown in FIG. 8, the learned model 36 in which the breed information 50 is an input and the information relating to the treatment policy for the dog breed represented by the breed information 50 is an output is generated, for each disease or injury. Then, the learning unit 42 stores the generated learned model 36 in the storage unit 22.

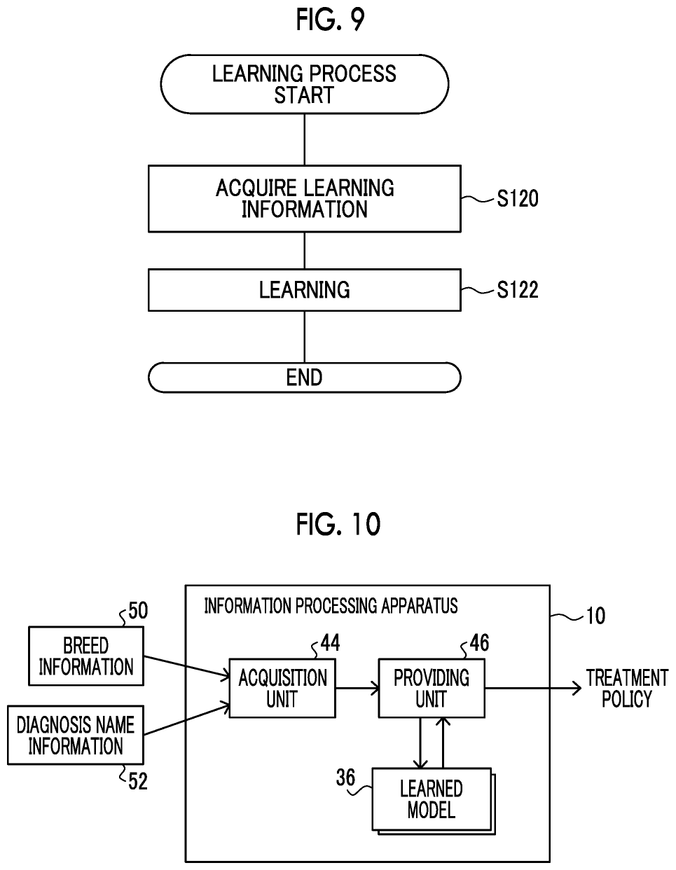

Next, an operation in a learning phase of the information processing apparatus 10 of the present embodiment will be described with reference to FIG. 9. As the CPU 20 executes the learning program 23B, the learning process shown in FIG. 9 is executed.

In Step S120 of FIG. 9, the learning unit 42 acquires the learning information 30 from the storage unit 22.

In the next Step S122, as described above, the learning unit 42 causes a model to be learned using the learning information 30 acquired in Step S120 as learning data for each disease or injury. Through this learning, the learning unit 42 generates the learned model 36 that outputs the information relating to the treatment policy on the basis of the breed information 50. Then, the learning unit 42 stores the generated learned model 36 in the storage unit 22. In a case where the process of Step S122 ends, the learning process ends.

Next, a functional configuration in an operation phase of the information processing apparatus 10 of the present embodiment will be described with reference to FIG. 10. As shown in FIG. 10, the information processing apparatus 10 of this embodiment includes an acquisition unit 44 and a providing unit 46. The CPU 20 functions as the acquisition unit 44 and the providing unit 46 by executing the treatment policy providing program 23C. The treatment record information collecting phase, the learning phase, and the operation phase may be executed by one information processing apparatus 10. Further, for example, the respective phases may be executed by different information processing apparatuses 10, and in this case, the regenerative medicine support system 1 is configured to comprise a plurality of information processing apparatuses 10.

The acquisition unit 44 acquires breed information 50 indicating a breed of a target animal that is a target for regenerative medicine and diagnosis name information 52 indicating a diagnosis name (a disease name or an injury name) of diagnosis performed by a veterinarian for the target animal, from the terminal device 12.

The providing unit 46 derives a treatment policy of the target animal on the basis of the breed information 50 acquired by the acquisition unit 44 and the learned model 36 learned in advance by the learning information 30, and provides the derived treatment policy to the terminal device 12. Specifically, the providing unit 46 inputs the breed information 50 acquired by the acquisition unit 44 to the learned model 36 for a disease or injury indicated by the diagnosis name information 52 acquired by the acquisition unit 44. The learned model 36 outputs information relating to the treatment policy according to the inputted breed information 50.

In addition, in a case where the information relating to the treatment policy input from the learned model 36 indicates that there is no treatment policy, the providing unit 46 derives a treatment policy for a closely related breed of the breed indicated by the breed information 50. Although cells are used for the regenerative medicine, in a case where cells derived from a breed different from the breed of the target animal that is the target of the regenerative medicine are used, an optimal treatment policy for the target animal cannot be obtained in some cases. Accordingly, in a case where the treatment record information 34 based on the breed of the target animal is not obtained, the providing unit 46 of the present embodiment provides a treatment policy of a closely related breed of the target animal. For example, in the case where the target animal is a mixed dog such as so-called “Chihuachshund”, the closely related breed is a breed of at least one of parents of the target animal. For example, in the case of the above-mentioned “Chihuachshund”, the providing unit 46 sets the closely related breed to at least one of “Chihuahua” or “Dachshund”. Further, the closely related breed is, for example, a breed that is determined to be closely related to the breed of the target animal on the basis of the gene, and as a specific example, a breed that has a relatively close relationship in a breed diagram of the breed. By using cells derived from the closely related breed for treatment in this way, it is possible to obtain a treatment policy using cells that are genetically similar, thereby obtaining an appropriate therapeutic strategy.

In addition, in a case where the information relating to the treatment policy input from the learned model 36 indicates that there is no treatment policy for the closely related breed, the providing unit 46 identifies a treatment policy with best (highly effective) treatment results with reference to the treatment record information 34 of the learning information 30.

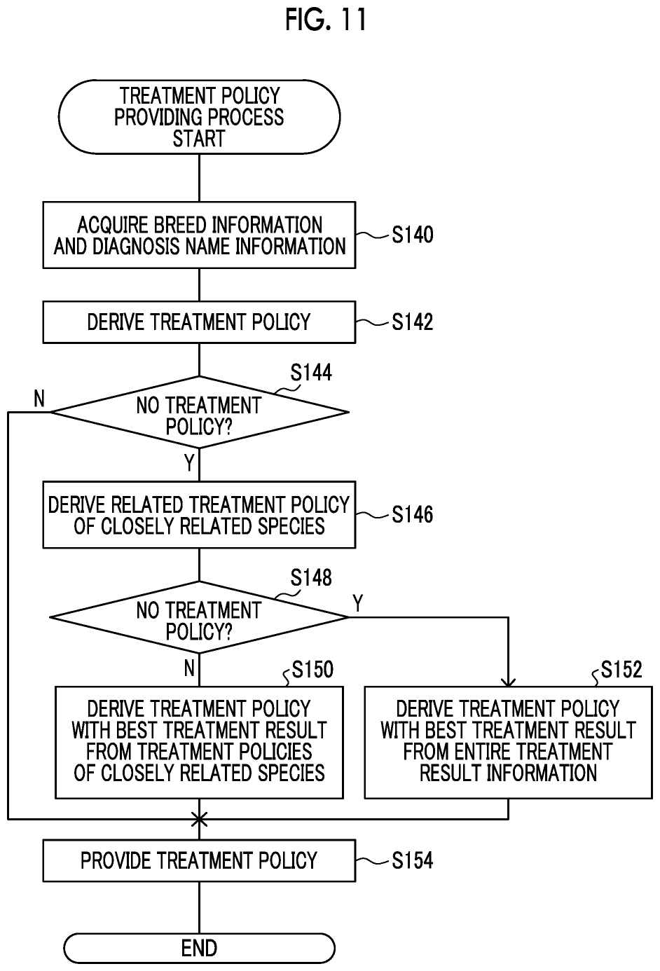

Next, an operation in the operation phase of the information processing apparatus 10 of the present embodiment will be described with reference to FIG. 11. As the CPU 20 executes the treatment policy providing program 23C, a treatment policy providing process shown in FIG. 11 is executed. The treatment policy providing process shown in FIG. 11 is executed, for example, in a case where the terminal device 12 gives a command for providing a treatment policy.

In Step S140 of FIG. 11, the acquisition unit 44 acquires the breed information 50 and the diagnosis name information 52 from the terminal device 12, as described above.

In the next Step S142, the providing unit 46 derives a treatment policy on the basis of the breed information 50, the diagnosis name information 52, and the learned model 36 input from the acquisition unit 44, as described above. Specifically, the providing unit 46 inputs the breed information 50 to the learned model 36 according to the disease or injury indicated by the diagnosis name information 52, and acquires information relating to the treatment policy output from the learned model 36.

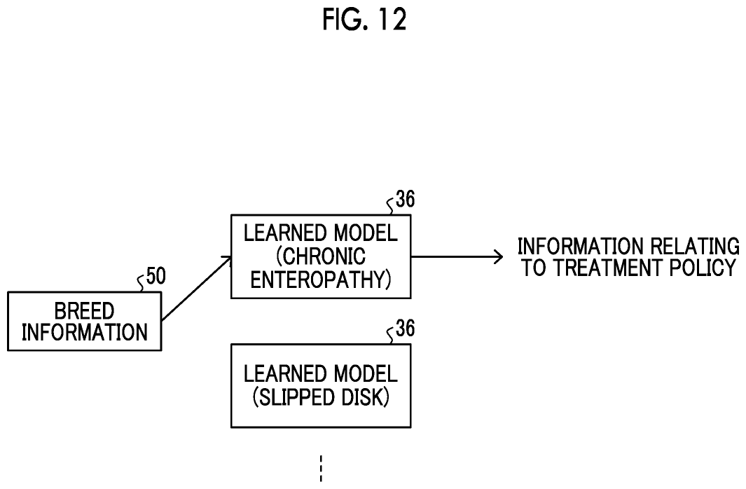

For example, as shown in FIG. 12, in a case where the disease or injury indicated by the diagnosis name information 52 is “chronic enteropathy”, the providing unit 46 inputs the breed information 50 to the learned model 36 for chronic enteropathy. The information relating to the treatment policy is output from the learned model 36.

In the next Step S144, the providing unit 46 determines whether the information relating to the treatment policy indicates that there is no treatment policy. In a case where the information relating to the treatment policy output from the learned model 36 is not the information indicating that there is no treatment policy, in other words, in a case where the information relating to the treatment policy is information indicating the treatment policy, the determination in Step S144 is negative, and then, the procedure proceeds to Step S154.

On the other hand, in a case where the information relating to the treatment policy output from the learned model 36 is the information indicating that there is no treatment policy, the determination in Step S144 is affirmative, and then, the procedure proceeds to Step S146. In Step S146, as described above, the providing unit 46 derives the treatment policy for the closely related breed of the breed indicated by the breed information 50, as in Step S142. In a case where there are a plurality of closely related breeds, the providing unit 46 may derive a treatment policy for at least one or more closely related breeds, but preferably derives treatment policies for a plurality of closely related breeds.

In the next Step S148, the providing unit 46 determines whether the information relating to the treatment policy for the closely related breed indicates that there is no treatment policy. In a case where the information relating to the treatment policy output from the learned model 36 is not the information indicating that there is no treatment policy, in other words, in a case where the information relating to the treatment policy is information indicating the treatment policy, the determination in Step S148 is negative, and then, the procedure proceeds to Step S150.

In Step S150, the providing unit 46 derives a treatment policy with the best treatment result from the treatment policies of the closely related breed, and then, the procedure proceeds to Step S154. Here, in the above Step S146, in a case where the treatment policy is derived only for one kind of closely related breed, the treatment policy derived in Step S146 may also be derived in Step S150, or Step S150 may be omitted.

On the other hand, in step 148, in a case where the information relating to the treatment policy output from the learned model 36 is the information indicating that there is no treatment policy, the determination in Step S148 is affirmative, and then, the procedure proceeds to Step S152.

In Step S152, the providing unit 46 derives the treatment record information 34 including treatment content information with the best treatment result indicated by treatment result information as a treatment policy, from the entire treatment record information 34, with reference to the treatment record information 34 of the learning information 30 stored in the storage unit 22.

In the next Step S154, the providing unit 46 provides the treatment policy derived in Step S150 or Step S152 to the terminal device 12. Specifically, the providing unit 46 outputs the treatment policy information indicating the treatment policy derived in Step S150 or Step S152 to the terminal device 12 through the network N. In a case where the treatment policy derived in Step S152 is provided to the terminal device 12, it is preferable that information indicating that the treatment record information 34 relating to breeds of the same and closely related breeds as the target animal is not obtained is also provided to the terminal device 12.

After the process of Step S154 ends, the present treatment policy providing process ends.

[Terminal Device]

An example of a hardware configuration of the terminal device 12 of the present embodiment will be described with reference to FIG. 13. As shown in FIG. 2, the terminal device 12 includes a central processing unit (CPU) 60, a memory 61 as a temporary storage area, and a non-volatile storage unit 62.

Further, the terminal device 12 includes a display unit 64 such as a liquid crystal display, an input unit 66 such as a keyboard and a mouse, and a network I/F 68 connected to the network N. The display unit 64 and the input unit 66 may be integrated as a touch panel display. The CPU 60, the memory 61, the storage unit 62, the display unit 64, the input unit 66, and the network I/F 68 are connected to the bus 69 to communicate with each other.

The storage unit 62 is realized by an HDD, an SSD, a flash memory, or the like. A treatment policy display program 63 is stored in the storage unit 62 that is a storage medium. The CPU 60 reads out the treatment policy display program 63 from the storage unit 62, expands the treatment policy display program 63 in the memory 61, and executes the expanded treatment policy display program 63. The treatment record information collecting program 23A, the treatment policy providing program 23C, and the treatment policy display program 63 of the present embodiment are examples of the regenerative medicine support program of the present disclosure.

Further, the electronic medical record information 70 is stored in the storage unit 62 of the present embodiment. In the terminal device 12, information relating to treatment performed at a veterinary hospital in which the terminal device 12 is installed is stored in the storage unit 62 as the electronic medical record information 70 in association with information relating to affected animals. The electronic medical record information 70 of the present embodiment includes at least information on treatment records of regenerative medicine performed at the veterinary hospital.

Next, an operation of the terminal device 12 of the present embodiment will be described with reference to FIG. 14. In a case where a veterinarian at a veterinary hospital desires a treatment policy in which a diagnosed animal is a target animal of regenerative medicine, the information processing apparatus 10 instructs execution of a treatment policy providing process from the terminal device 12. As an example, the terminal device 12 executes a treatment policy display process shown in FIG. 14 at a timing when the instruction of execution of the treatment policy providing process is output to the information processing apparatus 10. The treatment policy display process shown in FIG. 14 is executed by the CPU 60 executing the treatment policy display program 63.

In Step S200 of FIG. 14, the CPU 60 outputs, to the information processing apparatus 10, the breed information 50 indicating the breed of the target animal and diagnosis name information indicating a diagnosis name obtained by diagnosing the target animal. For example, in a case where a veterinarian who has medically cared a miniature dachshund diagnoses the miniature dachshund as having chronic enteropathy and intends to perform treatment by regenerative medicine, the breed information 50 indicating the miniature dachshund and the diagnosis name information 52 indicating the chronic enteropathy are output from the terminal device 12 to the information processing apparatus 10.

The breed information 50 and the diagnosis name information 52 output from the terminal device 12 are acquired by the information processing apparatus 10 in Step S140 (see FIG. 11) of the above-described treatment policy providing process. The information processing apparatus 10 derives the treatment policy using the learned model 36 as described above, which acquires the breed information 50 and the diagnosis name information 52, and provides the derived treatment policy to the terminal device 12.

Accordingly, in the next Step S202, the CPU 60 determines whether or not the treatment policy output from the information processing apparatus 10 has been input. The determination in Step S202 is negative until the treatment policy is input, and the determination in Step S202 is affirmative in a case where the treatment policy is input, and then, the procedure proceeds to Step S204.

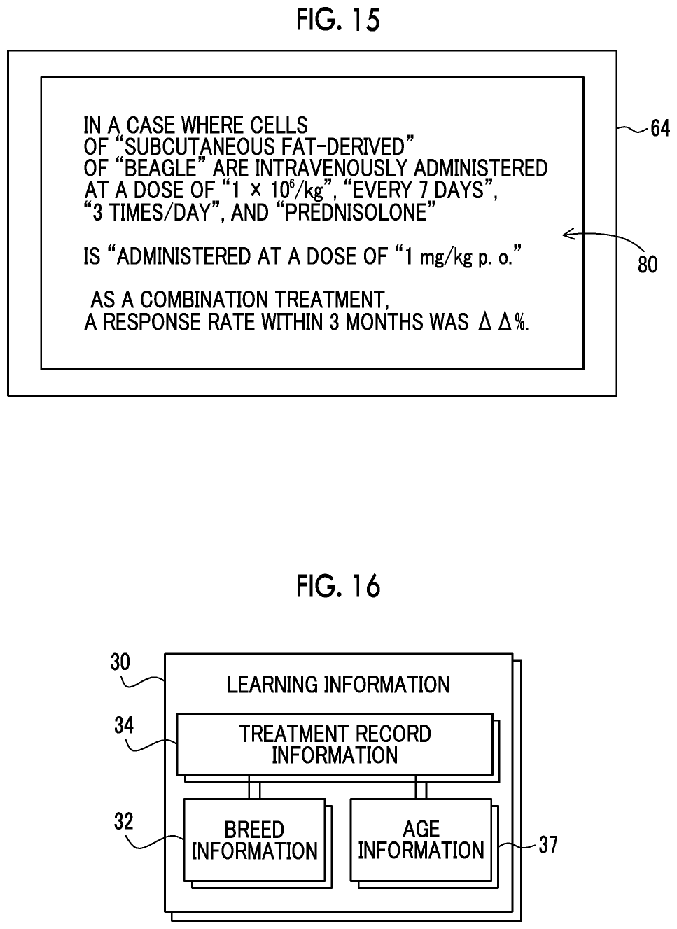

In Step S204, the CPU 60 causes the display unit 64 to perform a display based on the input treatment policy. FIG. 15 shows an example of a treatment policy 80 displayed on the display unit 64. FIG. 15 shows an example in which the treatment policy 80 indicating that cells of “subcutaneous fat-derived” of “Beagle” are intravenously administered at a dose of “1×10⁶/kg”, “every 7 days”, “3 times/day”, and “prednisolone” is “administered at a dose of “1 mg/kg p. o.” is displayed on the display unit 64, as a treatment policy for chronic enteropathy of the above-mentioned miniature dachshund. The display unit 64 also displays, as the treatment policy 80, that a treatment result of treatment according to the treatment policy is “a response rate within 3 months was ΔΔ%”. After Step S204 ends, the treatment policy display process ends. On the basis of the treatment policy 80 displayed on the display unit 64, the veterinarian can determine an actual treatment policy for the target animal.

Note that the learned model 36 may have a configuration shown in the following modified example, for example.

### Modified Example

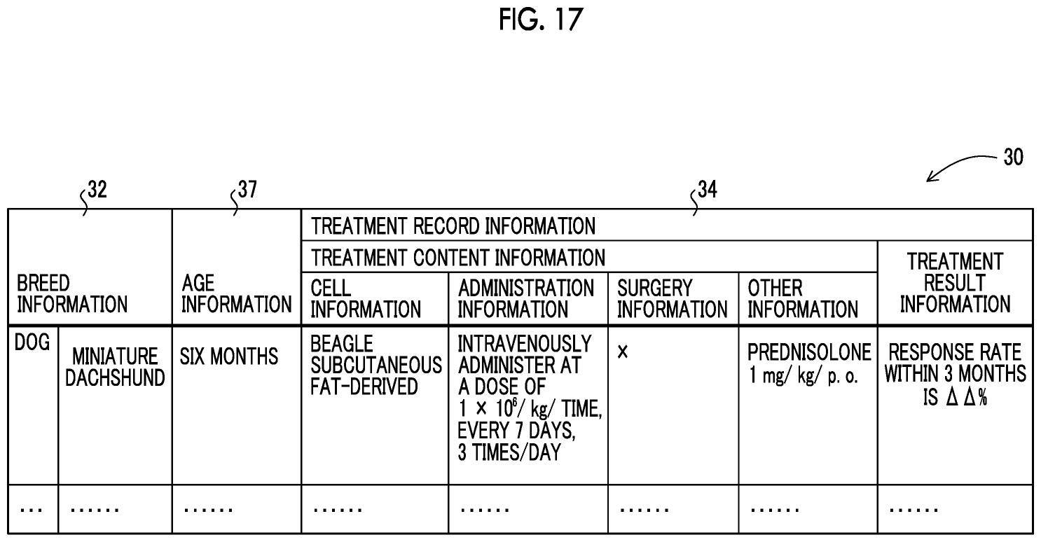

FIGS. 16 and 17 show an example of the learning information 30 used for learning of the learned model 36 of this modified example. As shown in FIGS. 16 and 17, the learning information 30 includes breed information 32, treatment record information 34, and age information 37.

The age information 37 is information that represents an age of an animal that is a target of treatment based on regenerative medicine, which is information that represents an elapsed time from birth. In this embodiment, for convenience, the age is expressed in the unit of years, but the age may be expressed as an elapsed time in months from birth, instead of the elapsed time in years from birth, that is, in the unit of months. For example, in the case of a dog or the like, which has a relatively fast growth, its body greatly changes according to the growth in the case of the age in the unit of years. Accordingly, in this embodiment, the information indicating the age in the unit of months is used as the age information 37. The age information 37 is not limited to the age in the unit of years and the age in the unit of months, and may be information indicating age in the unit of days, for example.

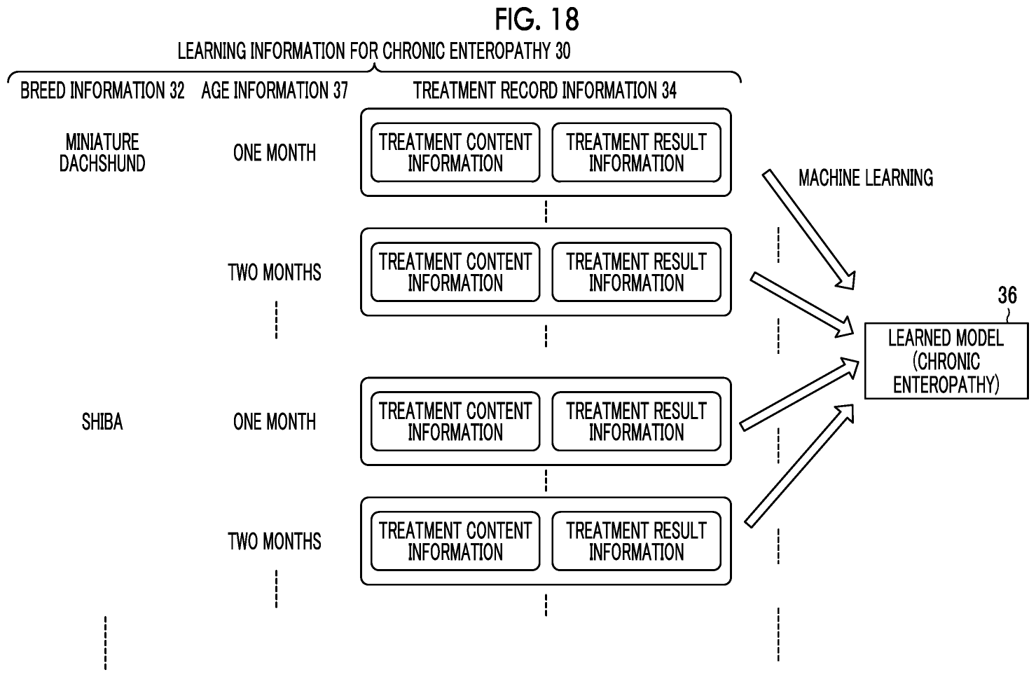

As an example, the learned model 36 of this modified example, as shown in FIG. 18, the learned model 36 is generated by machine learning using the learning information 30. For example, as shown in FIG. 18, the learned model 36 for chronic enteropathy is generated from the learning information 30 for chronic enteropathy, which includes the treatment record information 34 for which the dog breed indicated by the breed information 32 is “miniature dachshund” and the age indicated by the age information 37 is 1 month, the treatment record information 34 for which the dog breed indicated by the breed information 32 is “miniature dachshund” and the age indicated by the age information 37 is 2 months, the treatment record information 34 for which the dog breed indicated by the breed information 32 is “Shiba” and the age indicated by the age information 37 is 1 month, the treatment record information 34 for which the dog breed indicated by the breed information 32 is “Shiba” and the age indicated by the age information 37 is 2 months, and the like. An example of the learned model 36 includes a neural network model.

As shown in FIG. 18, the learning unit 42 of the present modified example generates a plurality of learned models 36 according to diseases or injuries through machine learning, in which the breed information 50 and the age information 54 representing the breed of the target animal are input and the information relating to the treatment policy is output, for each disease or injury.

More specifically, in a case where the breed information 50 is input, the learning unit 42 causes the model to be learned so that information relating to a treatment policy with the best treatment result (high treatment effect) for a dog breed indicated by the breed information 50 is output. Further, in a case where there is no treatment record information 34 in which the breed information 32 of the same breed as the breed indicated by the input breed information 50 and the age information 37 of the same age are associated with each other in the learning information 30 used for learning, the model is learned so that information indicating that there is no treatment policy is output. By this learning, the learned model 36 is generated for each disease or injury.

Furthermore, the learned model 36 is not limiting, and may employ other modified examples. For example, in the above-described embodiment, a configuration in which the learned model 36 is generated for each disease or injury and is used to derive the treatment policy is shown, but a configuration in which the learned models 36 corresponding to all diseases or injuries are generated and are used to derive the treatment policy may be used. In this case, for example, similar to the case where the breed information 50 and the age information 54 are used in combination in the above modified example, the breed information 50 and information indicating diseases or injuries may be used in combination.

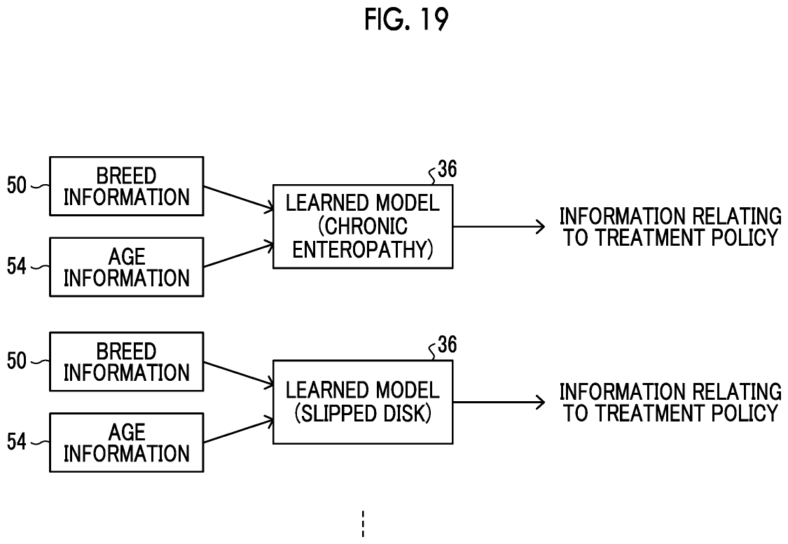

As an algorithm of the learning by the learning unit 42 described above, for example, backpropagation may be applied. Through the learning by the above-described learning unit 42, as shown in FIG. 19, as an example, the learned model 36 is generated in which the combination of the breed information 50 and the age information 54 is input and the information relating to the treatment policy for the animal of the breed indicated by the breed information 50 and the age indicated by the age information 54 is output, for each disease or injury. Then, the learning unit 42 stores the generated learned model 36 in the storage unit 22.

The acquisition unit 44 acquires the breed information 50 indicating the breed of the target animal that is the target for regenerative medicine, the diagnosis name information 52 indicating a diagnosis name of the target animal diagnosed by a veterinarian, and the age information 54 indicating the age of the target animal from the terminal device 12.

As described above, the providing unit 46 derives the treatment policy on the basis of the breed information 50, the diagnosis name information 52, the age information 54, and the learned model 36 input from the acquisition unit 44. Specifically, the providing unit 46 inputs the combination of the breed information 50 and the age information 54 to the learned model 36 according to the disease or injury indicated by the diagnosis name information 52, and acquires the information relating to the treatment policy output from the learned model 36.

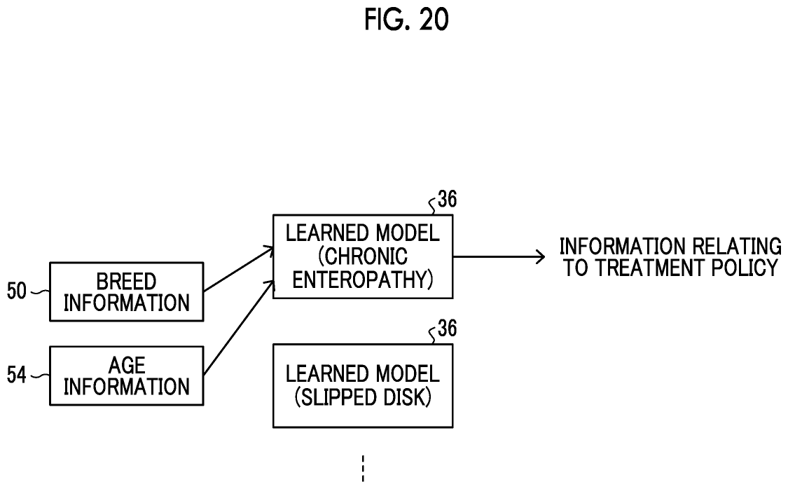

For example, as shown in FIG. 20, in a case where the disease or injury indicated by the diagnosis name information 52 is “chronic enteropathy”, the providing unit 46 inputs the combination of the breed information 50 and the age information 54 to the learned model 36 for chronic enteropathy. The information relating to the treatment policy is output from the learned model 36.

In the regenerative medicine support system 1 of the present embodiment, the breed information 50 and the age information 54 may be handled in combination. For example, in the operation of the information processing apparatus 10 of the present embodiment, the age information 54 may be acquired in addition to the breed information 50 and the diagnosis name information 52 in Step S140 (see FIG. 11) of the treatment policy providing process in the operation phase. Further, for example, in the operation of the terminal device 12 of the present embodiment, the age information 54 may be output in addition to the breed information 50 and the diagnosis name information 52 in Step S200 (see FIG. 14) of the treatment policy display process.

In the treatment policy providing process in the information processing apparatus 10 of the present modified example, in a case where the learned model 36 outputs the information relating to the treatment policy indicating that there is no treatment policy for the combination of the breed information 50 indicating the breed of the target animal and the age information 54 indicating the age, the following configuration may be used. A configuration in which in a case where it is determined in advance which information of the breed information 50 (breed) and the age information 54 (age) has a higher priority and the treatment policy is obtained from the learned model 36 for the above combination in which only the information with the higher priority matches, the information processing apparatus 10 provides the obtained treatment policy may be used. In the case of this configuration, considering that cells having genes closer to each other are used for treatment, it is preferable to give a higher priority to the breed information 50 among the breed information 50 and the age information 54.

As described above, in the present modified example, since the treatment policy according to the combination of the breed information 50 indicating the breed of the target animal and the age information 54 indicating the age can be obtained, it is possible to obtain an appropriate treatment policy according to the target animal that is a target for regenerative medicine.

As described above, the regenerative medicine support system 1 according to the above-described embodiment provides the CPU 20, and the storage unit 22 that stores a command executable by the CPU 20 in the information processing apparatus 10. The CPU 20 collects a plurality of pieces of treatment record information 34 including content of treatment based on regenerative medicine and a treatment result by the treatment for each breed of an animal that is a target of the treatment, obtains breed information indicating the breed of the target animal that is the target of the treatment, and provides a treatment policy of the target animal, which is derived on the basis of the treatment record information 34 in which the breed represented by the breed information 50 is the treatment target among the plurality of pieces of collected treatment record information 34.

According to the regenerative medicine support system 1, it is possible to provide the treatment policy of the target animal, which is derived on the basis of the treatment record information 34 representing a past treatment record, in which the breed of the target animal that is the target of the treatment based on the regenerative medicine is the treatment target. Accordingly, according to the regenerative medicine support system 1, it is possible to obtain an appropriate treatment policy according to the target animal that is the target of the regenerative medicine.

In addition, in a case where there is no treatment record information 34 in which the breed of the target animal is the treatment target, such as a case where the treatment having the breed of the target animal as the treatment target has not been performed in the past, the regenerative medicine support system 1 provides a treatment policy of the target animal, which is derived on the basis of the treatment record information 34 for a related breed whose genes are relatively close to those of the target animal. Accordingly, according to the regenerative medicine support system 1, even in a case where there is no treatment record information 34 in which the breed of the target animal is the treatment target, it is possible to obtain an appropriate treatment policy according to the target animal that is the target of the regenerative medicine.

In addition, in the above embodiment, a configuration in which that the information processing apparatus 10 derives a treatment policy using the learned model 36 has been described, but a treatment policy deriving method is not limited to the method using the learned model 36. For example, a configuration in which the regenerative medicine support system 1 has a database of regenerative medicine information equivalent to the learning information 30 and the providing unit 46 of the information processing apparatus 10 derives a treatment policy with reference to the regenerative medicine information database may be used.

Further, a hardware structure of a processing unit that executes various processes such as various functional units of the information processing apparatus 10 and the terminal device 12 in the regenerative medicine support system 1 according to the above-described embodiment, the following various processors may be used. As described above, in addition to the CPU that is a general-purpose processor that executes software (programs) to function as various processing units, the above-described various processors include a programmable logic device (PLD) that is a processor whose circuit configuration is changeable after manufacturing, such as a field-programmable gate array (FPGA), a dedicated electric circuit that is a processor having a circuit configuration that is exclusively designed to execute a specific process, such as an application specific integrated circuit (ASIC), or the like.

One processing unit may be configured by one of these various processors, or may be configured by a combination of two or more processors of the same type or different types (for example, a combination of a plurality of FPGAs or a combination of a CPU and an FPGA). Further, a plurality of processing units may be configured by one processor.

As an example in which the plurality of processing units is configured by one processor, firstly, as represented by a computer such as a client and a server, there is a configuration in which one processor is configured by a combination of one or more CPUs and software and the processor functions as a plurality of processing units. Secondly, as represented by a system on chip (SoC) or the like, there is a configuration in which a processor that realizes the functions of the entire system including a plurality of processing units by one integrated circuit (IC) chip is used. As described above, the various processing units are configured using one or more of the above various processors as a hardware structure.

Further, as a hardware structure of these various processors, more specifically, electric circuitry in which circuit elements such as semiconductor elements are combined may be used.

Further, in the above-described embodiment, a configuration in which the treatment record information collecting program 23A, the learning program 23B, and the treatment policy providing program 23C are stored (installed) in the storage unit 22 in advance and the treatment policy display program 63 is stored in the storage unit 62 in advance has been described, but the present invention is not limited thereto. Each of the treatment record information collecting program 23A, the learning program 23B, the treatment policy providing program 23C, and the treatment policy display program 63 may be provided in a form of being recorded on a recording medium such as a compact disc read only memory (CD-ROM), a digital versatile disc read only memory (DVD-ROM), a universal serial bus (USB) memory, or the like. Furthermore, each of the treatment record information collecting program 23A, the learning program 23B, the treatment policy providing program 23C, and the treatment policy display program 63 may be downloaded from an external device through a network.

Forceps and the following supplementary note are disclosed in the above-described embodiment.

(Supplementary Note 1)

A learning apparatus comprising: at least one processor; and a memory that stores a command executable by the processor, wherein the processor acquires learning information including breed information representing a breed of an animal that is a target of treatment based on a regenerative medicine, and treatment record information including content of the treatment and a treatment result based on the treatment, and generates a learned model in which information relating to a treatment policy of the treatment is output on the basis of the breed information representing the breed of the target animal that is the target of the treatment by causing the learning information to be learned as learning data.

What is claimed is:

1. A learning apparatus comprising:
at least one processor; and
a memory that stores a command executable by the processor,
wherein the processor collects a plurality of pieces of treatment record information including content of treatment based on a regenerative medicine and a treatment result by the treatment for each breed of an animal that is a target of the treatment, acquires breed information indicating the breed of the target animal that is the target of the treatment, and provides a treatment policy of the target animal, which is derived on the basis of the treatment record information in which the breed represented by the breed information is the treatment target among the plurality of pieces of collected treatment record information.

2. The regenerative medicine support system according to claim 1,
wherein the processor provides, in a case where the treatment record information in which the breed represented by the breed information is the treatment target is not included in the plurality of pieces of collected treatment record information, the treatment policy of the target animal, which is derived on the basis of the treatment record information in which a closely related breed of the breed represented by the breed information, instead of the breed represented by the breed information, is set as the treatment target.

3. The regenerative medicine support system according to claim 2,
wherein the closely related breed is a breed of at least one of parents of the target animal.

4. The regenerative medicine support system according to claim 2,
wherein the closely related breed is a breed that is determined to be closely related to the breed of the target animal on the basis of genes.

5. The regenerative medicine support system according to claim 1,
wherein the content of the treatment and the treatment policy include at least one of information relating to the type of cells used for the treatment, information relating to administration of the cells, or information relating to surgery.

6. The regenerative medicine support system according to claim 1,
wherein the processor performs the derivation of the treatment policy on the basis of a learned model learned in advance using the plurality of pieces of treatment record information and the breed represented by the breed information.

7. A regenerative medicine support system comprising:
an information processing apparatus including at least one processor and a memory that stores a command executable by the processor; and
a terminal device communicably connected to the information processing apparatus,
wherein the processor collects a plurality of pieces of treatment record information including content of treatment based on a regenerative medicine and a treatment result by the treatment for each breed of an animal that is a target of the treatment, acquires breed information indicating the breed of the target animal that is the target of the treatment, and provides a treatment policy of the target animal, which is derived on the basis of the treatment record information in which the breed represented by the breed information is the treatment target among the plurality of pieces of collected treatment record information, and
wherein the terminal device performs display based on the treatment policy provided from the information processing apparatus.

8. A regenerative medicine support method executed by a computer, the method comprising:
collecting a plurality of pieces of treatment record information including content of treatment based on a regenerative medicine and a treatment result by the treatment for each breed of an animal that is a target of the treatment;
acquiring breed information indicating the breed of the target animal that is the target of the treatment;
providing a treatment policy of the target animal, which is derived on the basis of the treatment record information in which the breed represented by the breed information is the treatment target among the plurality of pieces of collected treatment record information; and
performing display based on the provided treatment policy.

9. A non-transitory computer-readable storage medium storing a regenerative medicine support program causing a computer to execute:
a process of collecting a plurality of pieces of treatment record information including content of treatment based on a regenerative medicine and a treatment result by the treatment for each breed of an animal that is a target of the treatment;
a process of acquiring breed information indicating the breed of the target animal that is the target of the treatment; and
a process of providing a treatment policy of the target animal, which is derived on the basis of the treatment record information in which the breed represented by the breed information is the treatment target among the plurality of pieces of collected treatment record information.
